(19) United States
(12) Patent Application Publication
(10) Pub. No.: US 2019/0002537 A1
(43) Pub. Date: Jan. 3, 2019

(54) METHOD FOR ENRICHING A PREPARATION OF IMMUNOGLOBULINS WITH ANTI RSV IMMUNOGLOBULINS AND PREPARATION ENRICHED IN THIS WAY

(71) Applicant: LABORATOIRE FRANCAIS DU FRACTIONNEMENT ET DES BIOTECHNOLOGIES, Les Ulis (FR)

(72) Inventors: Gèrald PERRET, Anthony (FR); Abdessatar CHTOUROU, Elancourt (FR)

(21) Appl. No.: 15/765,383

(22) PCT Filed: Sep. 30, 2016
(86) PCT No.: PCT/FR2016/052512

§ 371 Date: Apr. 2, 2018

(30) Foreign Application Priority Data
Oct. 1, 2015 (FR) 1559329

(51) Int. Cl. C07K 16/10; A61P 11/00; A61P 31/14; C07K 16/06; C07K 14/135

(52) U.S. Cl. CPC C07K 16/1027; A61P 11/00; C07K 14/135; C07K 16/065; A61P 31/14

(57) ABSTRACT

Disclosed is a method for preparing an immunoglobulin (Ig) concentrate useful for treating RSV infection, including a step consisting in subjecting an Ig composition derived from blood plasma to affinity chromatography utilizing an RSV-specific ligand. In a particular embodiment, the RSV-specific ligand is an RSV F protein, preferentially in prefusion conformation, or a variant or an antigenic fragment thereof.

The present invention relates to the enrichment of a preparation of immunoglobulins (Ig) derived from human plasma, with immunoglobulins capable of recognizing, and advantageously of neutralizing, the respiratory syncytial virus (RSV). The invention also provides an Ig composition thus enriched, useful in the treatment of RSV infection.

### BACKGROUND OF THE INVENTION

The respiratory syncytial virus (RSV) is a widespread respiratory tract infectious agent. RSV usually induces a benign infection in immunocompetent individuals, but is the most important cause of bronchiolitis and pneumonia in infants and young children. Currently, there is no effective vaccine available to prevent RSV infection. Although anti-viral drugs are proposed to treat RSV infections, their efficacy in children is debatable.

RespiGam®, a preparation of pooled hyperimmune normal immunoglobulins which contains a high titre of immunoglobulins against RSV, was used as an alternative immunoprophylactic approach. Nevertheless, RespiGam® is a hyperimmune immunoglobulin concentrate, it is thus prepared from plasma of individuals selected for their high titre of anti-RSV immunoglobulins, and by using a conventional immunoglobulin fractionation process. Thus, although comprising more anti-RSV immunoglobulins than a conventional immunoglobulin concentrate, RespiGam® does not comprise more than 1% anti-RSV immunoglobulins. Moreover, donor selection limits the production volume.

The emergence on the market of a highly specific monoclonal antibody, palivizumab, led to the withdrawal of the hyperimmune immunoglobulin composition RespiGam® from the market. However, the cost and the agent-specific nature of the monoclonal antibodies also make this solution unsatisfactory.

In that context, Gupta et al., PLOS One, 2013, 8(7):1-5 showed the presence of anti-RSV immunoglobulins in intravenous immunoglobulin G (IgIV) preparations, obtained from pooled plasma from several thousand healthy blood donors. To that end, Gupta et al. retained RSV-specific immunoglobulins by passing through affinity matrices bearing recombinant RSV G protein. The presence of anti-RSV immunoglobulins in these IgIV preparations is explained by the high prevalence of the virus throughout the world. Nevertheless, Sastre et al. showed that such IgIV preparations had a low RSV-neutralizing activity.

There is thus a need for a method for producing immunoglobulin preparations hyper-enriched with immunoglobulins capable of neutralizing the respiratory syncytial virus (RSV).

### SUMMARY OF THE INVENTION

The inventors now propose to subject immunoglobulin (Ig) preparations derived from human plasma or from blood plasma fractions to affinity chromatography utilizing an RSV protein as affinity ligand, to produce immunoglobulin preparations hyper-enriched with immunoglobulins capable of recognizing, and advantageously of neutralizing, the respiratory syncytial virus (RSV). Preferably the RSV F protein, advantageously stabilized in prefusion or postfusion conformation, is used as affinity chromatography ligand.

Particular conformations of the RSV F protein, and especially the prefusion conformation, advantageously make it possible to more specifically retain the anti-RSV immunoglobulins having neutralizing activity.

More precisely, the invention relates to a method for preparing an immunoglobulin (Ig) concentrate useful for treating RSV infection comprising a step consisting in subjecting an Ig composition derived from blood plasma to affinity chromatography utilizing an RSV-specific ligand.

Said RSV-specific ligand can be an RSV protein, in particular the RSV G, SH and F proteins, or a variant of these proteins or an antigenic fragment of these proteins or of their variants.

In a particular embodiment the RSV-specific ligand is an RSV F protein, preferentially in prefusion conformation, or a variant, or an antigenic fragment thereof or one of its variants.

In a particular embodiment, the RSV-specific ligand, for example the RSV F protein, preferably in prefusion conformation, or an antigenic fragment thereof, is bound to the affinity matrix by a covalent bond, either directly or via a spacer.

Advantageously the RSV protein can be a variant protein, for example it can be an RSV F protein that contains the mutations S155C, S290C, S190F and/or V207L.

Another object of the invention is an Ig concentrate enriched with anti-RSV Ig, obtained by the above method, particularly useful in the treatment of RSV infection.

### BRIEF DESCRIPTION OF THE FIGURES

FIG. 1, extracted from McLellan et al., shows a model of the F protein in prefusion conformation;

FIG. 2, also extracted from McLellan et al., illustrates the transition from the prefusion conformation (at left) to the postfusion conformation (at right) of antigenic site Ø;

FIG. 3 represents a curve of percent infection as a function of the log of antibody concentration;

FIG. 4 represents a curve of percent infection as a function of the log of antibody concentration;

FIG. 5 represents the effect of the F protein ligand on serum neutralization of IVIGs.

### DETAILED DESCRIPTION OF THE INVENTION

RSV:

The respiratory syncytial virus (RSV) is a widespread respiratory tract infectious agent. RSV usually induces a benign infection, either asymptomatic or with mild symptoms (cold), in immunocompetent individuals, but which can be responsible for several infectious episodes per year in the same individual, potentially causing absences from school or work.

Moreover, this virus is an important factor in hospital infections in immunocompromised patients, in patients awaiting or following transplant, in infants or young children or in the elderly. RSV is the most important cause of bronchiolitis and pneumonia in infants and young children, and is largely responsible for respiratory infections in elder care facilities.

Within meaning of the present invention, “RSV” or “respiratory syncytial virus” refers both to the free viral particle present in the patient's biological fluids and to the viral particle having fused with the patient's host cell and giving rise to the formation of syncytia.

The RSV Proteins:

The respiratory syncytial virus is an RNA virus comprising 10 genes encoding 11 proteins including:
the M protein, matrix protein necessary for assembly of the viral particle;
the M2 protein, 2^{nd} matrix protein, also required for transcription. M2 contains CD8 epitopes;
the P protein;
the N protein of the nucleocapsid which will combine with the viral genome;
the RNA polymerase L protein;
the envelope proteins: the SH protein, the G protein (highly glycosylated transmembrane glycoprotein responsible for attachment to the host cell), the F protein (transmembrane glycoprotein responsible for fusion with the host membrane, for entry of the virus into the cell, and for formation of syncytia).

Within meaning of the present invention, “RSV protein” refers both to one of the 11 proteins encoded by the genome of the virus in its native form and to one of their variants or their antigenic fragments.

Among these proteins, some are advantageously present on the RSV surface, playing a role in the mechanisms of infection, especially during recognition, during binding of the viral particle to its host cell, during fusion of the viral particle with its host cell, and during propagation of the virus from host cell to host cell. These proteins, having the advantage of being accessible to the immune system, are in particular:
the SH protein,
the G protein,
the F protein.

The accessibility of these surface proteins can be constitutional or induced. In particular, the accessibility of the protein can be modified by a change in conformation, revealing all or part of the epitopes that were theretofore inaccessible to the immune system.

Within meaning of the present invention, the expression “RSV surface proteins” refers to one or a combination of the proteins accessible to the immune system, in their native form regardless of their conformation, one of their variants, or of their antigenic fragments. The RSV surface proteins are present on the RSV membrane, both on the free viral particle present in the patient's biological fluids and on the viral particle having fused with the patient's host cell and giving rise to the formation of syncytia.

The term “variant” includes any sequence subjected to one or more substitutions, additions and/or deletions, with no substantial alteration of the protein's conformation and/or stability. The variants considered have a sequence identity of at least 80%, preferably of at least 85%, more preferably of at least 90%, more preferably of at least 95%.

Advantageously, the antigenic fragments allow the attachment of anti-RSV neutralizing immunoglobulins. Preferably, they are fragments of at least 10 amino acids, more preferably at least 20, 30, 40, or 50 amino acids, which comprise one or more RSV protein epitopes. The RSV protein antigenic fragments used are advantageously peptides offering linear epitopes, unconstrained by the three-dimensional structure, which can be exposed during conformational changes of the protein. The RSV protein antigenic fragments used are advantageously also peptides offering conformational epitopes, which can be exposed during conformational changes of the protein.

The RSV F Protein:

The RSV F protein is a surface glycoprotein which mediates the phenomenon of virion fusion with the membrane of the cell it infects, allowing the virus to enter the cell cytoplasm. The F protein has a conformational diversity. Fusion proceeds from the difference in folding energy between two states: a metastable state adopted before the virus-cell interaction (the “prefusion” state), and a stable state formed after fusion (the “postfusion” state). Advantageously, the RSV F protein used according to the invention reveals epitopes allowing the attachment of anti-RSV neutralizing immunoglobulins.

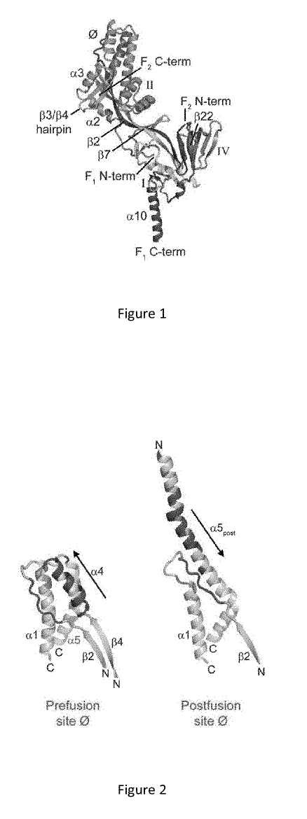

In a particular embodiment of the invention, the RSV F protein used is in prefusion conformation. This conformation was modelled in the article by McLellan et al., Science, 2013, 340:1113-1117. The antigenic site of the RSV F protein, preferentially recognized by neutralizing antibodies, is designated therein “antigenic site Ø” (see FIGS. 1 and 2).

The native sequence of the RSV F protein (SEQ ID NO: 1) is listed in the GenBank database, under accession number AHB33455.

SEQ ID NO: 1:

mellihrssa ifltlainal yltssqnite efyqstcsav srgylsalrt gwytsvitielsniketkcn gtdtkvklik qeldkyknav telqllmqnt paannrarre apqymnytinttkninvsis kkrkrrflgf llgvgsaias giayskvlhl egevnkikna llstnkavvslsngvsvlts kvldlknyin nqllpivnqq scrisnietv iefqqknsrl leitrefsvnagvttplsty mltnsellsl indmpitndq kklmssnvqi vrqqsysims iikeevlayvvqlpiygvid tpcwklhtsp lcttnikegs nicltrtdrg wycdnagsys ffpqadtckvqsnrvfcdtm nsltlpsevs lcntdifnsk ydckimtskt disssvitsl gaivscygktkctasnknrg iiktfsngcd yvsnkgvdtv svgntlyyvn klegknlyvk gepiinyydplvfpsdefda sisqvnekin qslafirrsd ellhnvntgk sttnimitai iiviivvllsliaiglllyc kakttpvtls kdqlsginni afsk

Within the meaning of the present invention, the term “RSV F protein” refers to the native form of sequence SEQ ID NO: 1, or to one of its variants.

The term “variant” includes any sequence subjected to one or more substitutions, additions and/or deletions, with no substantial alteration of the protein's conformation and/or stability. The variants considered have a sequence identity of at least 80%, preferably of at least 85%, more preferably of at least 90%, more preferably of at least 95% with the native sequence.

Variants of the F protein are, for example, described in the GenBank database, under accession numbers AAB59858 (SEQ ID NO: 2) and P11209 (SEQ ID NO: 3).

SEQ ID NO: 2 (AAB59858):

mellilkana ittiltavtf cfasgqnite efyqstcsav skgylsalrt gwytsvitielsnikenkcn gtdakvklik qeldkyknav telqllmqst pptnnrarre lprfmnytlnnakktnvtls kkrkrrflgf llgvgsaias gvayskvlhl egevnkiksa llstnkavvslsngvsvlts kvldlknyid kqllpivnkq scsisnietv iefqqknnrl leitrefsvnagvttpvsty mltnsellsl indmpitndq kklmsnnvqi vrqqsysims iikeevlayvvqlplygvid tpcwklhtsp lcttntkegs nicltrtdrg wycdnagsys ffpqaetckvqsnrvfcdtm nsltlpsein lcnvdifnpk ydckimtskt dvsssvitsl gaivscygktkctasnknrg iiktfsngcd yvsnkgmdtv svgntlyyvn kqegkslyvk gepiinfydplvfpsdefda sisqvnekin qslafirksd ellhnvnagk sttnimitti iiviivillsliavglllyc karstpvtls kdqlsginni afsn

SEQ ID NO: 3 (P11209):

melpilktna itailaavtl cfassqnite efyqstcsav skgylsalrt gwytsvitielsnikenkcn gtdakvklik qeldkyksav telqllmqst patnnrarre lprfmnytlnntkntnvtls kkrkrrflgf llgvgsaias giayskvlhl egevnkiksa llstnkavvslsngvsvlts kvldlknyid kqllpivnkq scsisnietv iefqqknnrl leitrefsvnagvttpvsty mltnsellsl indmpitndq kklmsnnvqi vrqqsysims iikeevlayvvqlplygvid tpcwklhtsp lcttntkegs nicltrtdrg wycdnagsys ffplaetckvqsnrvfcdtm nsltlpsevn lcnidifnpk ydckimtskt dvsssvitsl gaivscygktkctasnkdrg iiktfsngcd yvsnkgvdtv svgntlyyvn kqegkslyvk gepiinfydplvfpsdefda sisqvnekin qslafirksd ellhnvnagk sttnimitti iiviivillsliavglllyc karstpvtls kdqlsginni afsn

In particular, included are the variants designated “DS” (containing the double mutation 5155C, 5290C), “Cav1” (containing the double mutation 5190F, V207L), or “DS-Cav1” (containing the four mutations S155C, S290C, S190F, V207L), as described in the article by McLelland et al., 2013, SCIENCE, 342: 592-598.

In a preferred embodiment, the variant designated “DS-Cav1” containing the mutations S155C, S290C, S190F, V207L is used.

Also included is the variant designated “FcN_{2C-C}” (containing the 4 mutations L481C, D489C, 5509C, D510C) as described in the article by Magro et al., 2012, PNAS, vol. 109, no. 8: 3089-3094.

The RSV F protein, without its prefusion conformation, can be prepared by all conventional purification techniques, by peptide synthesis and especially by chemical synthesis, by genetic engineering, etc.

Preferably, a recombinant F protein is used, which can be obtained by a conventional recombinant protein production process, comprising transferring an expression vector to a host cell, under conditions allowing expression of the recombinant protein encoded by the vector, and collecting the protein thus produced. The expression vector can be prepared according to the methods commonly used by the person skilled in the art and can be introduced into the host cell by standard methods such as lipofection, electroporation, heat-shock, etc. The host cell can especially be a bacterium, a yeast, a moss, a fungus, a plant cell or a mammalian cell.

The RSV F protein antigenic fragments can be used alternatively as ligands for affinity chromatography. Advantageously, the antigenic fragments allow the attachment of anti-RSV neutralizing immunoglobulins. Preferably they are fragments of at least 10 amino acids, more preferably at least 20, 30, 40, or 50 amino acids, which comprise one or more RSV F protein epitopes.

The RSV F protein antigenic fragments used are advantageously peptides offering linear epitopes, unconstrained by the three-dimensional structure, which are normally exposed on the F protein in prefusion conformation, but which are not exposed to the solvents in postfusion conformation.

The RSV F protein antigenic fragments used are advantageously also peptides offering conformational epitopes, which are normally exposed on the F protein in prefusion conformation, but which are not exposed to the solvents in postfusion conformation.

Included in particular are the fragments designated “F24-136” (F fragment derived from the F2 chain), “F164-315” (fragment consisting of the N-terminal 2/3 of the F1 chain), “F283-402” (central segment of the F1 chain) and “F403-524” as described in the article by Sastre et al., Vaccine 23 (2004) 435-443. Also included are the fragments corresponding to the peptides designated “F167-201” (heptad repeat A fragment), “F235-275” (antigenic site II fragment) and “F478-512” (heptad repeat B fragment) as described in the article by Sastre et al., Vaccine 23 (2004) 435-443.

In a preferred embodiment, the fragments designated “F24-136”, “F164-315”, “F283-402”, “F403-524”, “F167-201”, “F235-275”, and/or “F478-512” are used.

In another particular embodiment of the invention, the fragments corresponding to the heptad repeat A and/or heptad repeat B regions of the F protein are used, alone or in combination.

In another embodiment of the invention, the RSV F protein antigenic fragments used are a combination of several peptides offering linear epitopes, unconstrained by the three-dimensional structure, which are normally exposed on the F protein in prefusion conformation, but which are not exposed to the solvents in postfusion conformation.

The RSV G Protein:

The RSV G protein is a surface transmembrane glycoprotein allowing the virus to bind to the host cell. It interacts with receptor CX3CR1 of the host cell to modulate the immune response and to facilitate the infection. The G protein comprises 289 to 299 amino acids (32-33 kDa), depending on the strain, and is palmitoylated. Highly glycosylated, it comprises 30-40 O-glycosylations and 4-5 N-glycosylations. The glycosylation and thus the size of the G protein depend on the cell type in which it is produced: 80-100 kDa in immortalized cell lines, but 180 kDa in primary cultures of amniotic epithelial cells. The G protein comprises a central conserved domain (130-230) with a highly conserved portion of 13 amino acids (164-176), a “cysteine noose” with 2 disulphide bridges (C173, C176, C182, C186), and a CX3C motif of 5 amino acids (C182-XXX-C186). RSV strain B further comprises a repeat domain of 20 amino acids in the second mucin-like domain, whereas RSV strain A comprises in the same region a repeat domain of 24 amino acids. Advantageously, the RSV G protein used according to the invention possesses epitopes allowing the attachment of anti-RSV neutralizing immunoglobulins.

In a particular embodiment of the invention, the RSV G protein used is derived from RSV strain A and designated GA.

The native sequence of the RSV GA protein (SEQ ID NO: 4) is listed in the GenBank database, under accession number P27022.

SEQ ID NO: 4

msknkdqrta ktlertwdtl nhllfisscl yklnlksvaq itlsilamii stsliivaii fiasanhkit stttiiqdat nqiknttpty ltqnpqlgis psnpsditsl ittildsttp gvkstlqstt vgtknttttq aqpnkpttkq rqnkppskpn ndfhfevfnf vpcsicsnnp tcwaickrip nkkpgkrttt kptkkptpkt tkkgpkpqtt kskeapttkp teeptinttk tniittllts nttrnpelts qmetfhstss egnpspsqvs itseypsqps sppntpr

In another particular embodiment of the invention, the RSV G protein used is derived from RSV strain B and designated GB.

The native sequence of the RSV GB protein (SEQ ID NO: 5) is listed in the GenBank database, under accession number 036633.

SEQ ID NO: 5

mskhknqrta rtlektwdtl nhlivisscl yrinlksiaq ialsvlamii stsliiaaii fiisanhkvt lttvtvqtik nhteknitty ltqvppervs sskqptttsp ihtnsattsp ntksethhtt aqtkgrttts tqtnkpstkp rlknppkkpk ddyhfevfnf vpcsicgnnq lcksicktip snkpkkkpti kptnkpttkt tnkrdpktpa kttkketttn ptkkptlttt erdtstsqst vldtttleht iqqqslhstt pentpnstqt ptasepstsn stqntqsha

Within the meaning of the present invention, the term “RSV G protein” refers to the native form of sequence SEQ ID NO: 4 or SEQ ID NO: 5, or to one of its variants.

In a particular embodiment of the invention, variants of the G protein are used.

The term “variant” includes any sequence subjected to one or more substitutions, additions and/or deletions, with no substantial alteration of the protein's conformation and/or stability. The variants considered have a sequence identity of at least 80%, preferably of at least 85%, more preferably of at least 90%, more preferably of at least 95% with the native sequence.

The RSV G protein can be prepared by all conventional purification techniques, by peptide synthesis and especially by chemical synthesis, by genetic engineering, etc.

Preferably, a recombinant G protein is used, which can be obtained by a conventional recombinant protein production process, comprising transferring an expression vector to a host cell, under conditions allowing expression of the recombinant protein encoded by the vector, and collecting the protein thus produced. The expression vector can be prepared according to the methods commonly used by the person skilled in the art and can be introduced into the host cell by standard methods such as lipofection, electroporation, heat-shock, etc. The host cell can especially be a bacterium, a yeast, a moss, a fungus, a plant cell or a mammalian cell.

The antigenic fragments of the RSV G protein can be used alternatively as ligands for affinity chromatography. Advantageously, the antigenic fragments allow the attachment of anti-RSV neutralizing immunoglobulins. Preferably they are fragments of at least 10 amino acids, more preferably at least 20, 30, 40, or 50 amino acids, which comprise one or more RSV G protein epitopes. Advantageously, the antigenic fragments are comprised of all or part of the central conserved region of the G protein (164-186).

The RSV G protein antigenic fragments used are advantageously peptides offering linear epitopes, unconstrained by the three-dimensional structure, which are normally exposed on the G protein.

The RSV G protein antigenic fragments used are advantageously also peptides offering conformational epitopes, which are normally exposed on the G protein.

Included in particular are the fragments “G2Na” and “G2Nb” (fragments 130-230) as described in Nguyen et al., PLoS ONE, March 2012, Volume 7, Issue 3 or the fragments “Gs” as described in Sastre et al., Vaccine 23 (2004) 435-444 in particular in FIG. 6.

In a preferred embodiment, the fragments designated “G2Na” and/or “G2Nb” and/or “Gs” are used.

In another particular embodiment of the invention, the fragments corresponding to the conserved regions of the G protein are used, alone or in combination.

In another embodiment of the invention, the RSV G protein antigenic fragments consist of one or more peptides corresponding to an exposed area of the virus.

In another embodiment of the invention, the RSV G protein antigenic fragments used are a combination of several peptides offering linear epitopes, unconstrained by the three-dimensional structure, advantageously of several peptides of strains A and/or B.

In a particular embodiment of the invention, the RSV G protein or its variants or its fragments is/are glycosylated, preferably sufficiently glycosylated to maintain a minimal conformation of the epitope(s) allowing their recognition by the anti-RSV immunoglobulins.

The Chromatographic Support:

The method of the invention employs affinity chromatography. This chromatography utilizes a matrix comprising a support based on polymer particles, and is preferably in gel or resin form. These polymer particles are preferably spherical or oblong in shape, in particular they can be beads. The polymer can be natural or unnatural, organic or inorganic, crosslinked or uncrosslinked. The polymer is preferably an organic polymer, preferably crosslinked.

In a preferred embodiment, the polymer is cellulose, and the particles are preferably porous cellulose beads. Other possible types of polymers include agarose, dextran, polyacrylates, polystyrene, polyacrylamide, polymethacrylamide, copolymers of styrene and divinylbenzene, or mixtures of these polymers.

The particles can provide a chromatography medium which can be used to fill a column, for example.

An RSV protein, or an antigenic fragment thereof, is grafted onto the support, either directly or via a spacer, which covalently binds the ligand to the particles of the chromatographic support.

In a preferred embodiment of the invention, the protein grafted onto the support is an RSV surface protein, for example the RSV F protein or the RSV G protein, or an antigenic fragment thereof, either directly or via a spacer, which covalently binds the ligand to the particles of the chromatographic support.

In a particular embodiment of the invention, the grafted support bears several different ligands. The support can thus be grafted with several different proteins and/or several different antigenic fragments of RSV, advantageously several RSV surface proteins or their fragments, in order to obtain an anti-RSV immunoglobulin concentrate having various antigenic targets.

In a particular embodiment of the invention, the grafted support thus bears a mixture of ligands consisting of:
F protein in prefusion conformation, and/or
F protein in postfusion conformation, and/or
GA protein, and/or
GB protein

and/or their respective fragments or variants.

In an advantageous embodiment of the invention, the grafted support thus bears a mixture of ligands consisting of:
F protein in prefusion conformation, advantageously the ligand hRSV-F 11049-V08B, and
GA protein, advantageously the ligand 11070-V08H2, SB, and
GB protein, advantageously the ligand 13029-VO8H, SB.

The support can also be grafted with one or more RSV proteins and one or more proteins derived from one or more other infectious agents in order to obtain a concentrate comprising both anti-RSV immunoglobulins (optionally with various antigenic targets) and immunoglobulins directed against another infectious agent.

Advantageously in this embodiment, each protein corresponds to at least 30 wt %, or at least 50 wt % of the antigens grafted onto the support.

A particle can bear several spacers.

The bond between the ligand and the spacer can be, for example, an amide bond.

The spacer typically comprises at least one C, O, N, or S atom.

The ligands are chemically immobilized by covalent bonds between the particles and the spacer, and between the spacer and the ligand. This immobilization can be achieved conventionally by the person skilled in the art.

In a preferred embodiment, the particle bears an —NH—R1-COOH arm. Preferably it is alpha-aminocaproic acid (where R1 is a pentyl group).

Conventionally, the particle can be activated by using bifunctional reagents such as epichlorohydrin, epibromohydrin, dibromo- and dichloropropanol, dibromobutane, ethylene glycol diglycidyl ether, butanediol diglycidyl ether, divinyl sulphone, allyl glycidyl ether, and allyl bromide. The bifunctional reagent is capable of reacting with both the particles and the —NH—R1-COOH arm. Allyl heterofunctional compounds, such as allyl bromide, are preferred bifunctional reagents and make it possible to obtain an activated matrix. For some solid supports, such as cellulose, composites containing a hydrogel or other materials with hydroxyl groups, it is advantageous to deprotonate the hydroxyl groups with a hydroxide source, for example, before reaction with a bifunctional reagent.

The matrix in gel form is prepared by conventional addition of a buffer to the polymer particles carrying the ligands, as is known to the person skilled in the art, so as to obtain a matrix in gel form, suitable for affinity chromatography.

The matrix as defined herein is useful in affinity chromatography binding anti-RSV immunoglobulins. The matrix is particularly useful in mixed-bed affinity chromatography. Such a matrix advantageously comprises several different antigens from RSV and is thus capable of binding anti-RSV immunoglobulins directed against several different epitopes. Such a matrix can also advantageously comprise several different antigens derived from different infectious agents, and is thus capable of binding both anti-RSV immunoglobulins and immunoglobulins directed against another selected infectious agent.

To that end, the matrix can be introduced into a chromatography column. On an industrial scale, the column can contain from 1 to 150 litres, even 250 to 500 L, if necessary. In the case of a pilot-scale implementation, columns from 1 to 50 cm in height can be used, the diameter then being adapted to the height of the column used. The matrix volume can also be adjusted to meet the requirements of an industrial process, especially to be adapted to the product volumes to be processed.

The anti-RSV immunoglobulins present in the plasma or the plasma fraction are bound to the matrix, and the adsorbed product is eluted and collected, enriched with anti-RSV immunoglobulins.

In a particular embodiment of the invention, the matrix is grafted with ligands of F protein type, in postfusion or prefusion conformation.

In an embodiment of the invention, the RSV F protein remains in and/or returns to its prefusion conformation during all the steps of preparation and use of the matrix: grafting, washing, equilibration, loading, washing, elution, regeneration, sanitization.

In a preferred embodiment of the invention, the RSV F protein is grafted onto the matrix in its prefusion conformation. The grafting method and the possible choice of spacer are thus adapted by the person skilled in the art in order to maintain the RSV F protein in the prefusion conformation.

In another particular embodiment of the invention, the matrix is grafted with ligands of G protein type derived from RSV strain A and/or strain B.

Advantageously, the conditions for executing the affinity chromatography according to the invention are adapted by the person skilled in the art so as to guarantee that the matrix according to the invention can be reused, all while maintaining elution conditions that do not degrade the product of interest.

Plasma or Plasma Fraction:

The starting plasma or plasma fraction subjected to the enrichment method of the invention advantageously consists of plasma from blood plasmas or pooled blood plasmas from mammalian subjects (human or nonhuman) or of a fraction of this plasma, i.e., any part or subpart of the plasma, having undergone one or more purification steps.

In a particular embodiment, the starting plasma or plasma fraction subjected to the enrichment method of the invention advantageously consists of plasma from blood plasmas or pooled blood plasmas from nonhuman mammalian subjects, male and/or female, advantageously goats, sheep, bison, buffalo, camels, llamas, mice, rats, cattle, pigs, rabbits, horses. The nonhuman mammal is advantageously transgenic and has been previously exposed to RSV in order to produce anti-RSV human immunoglobulins in its plasma.

In another particular embodiment of the invention, the starting plasma or plasma fraction subjected to the enrichment method of the invention advantageously consists of plasma from pooled blood plasmas from normal human subjects or of a fraction of this plasma, i.e., any part or subpart of the plasma, having undergone one or more purification steps.

The usable plasma fractions thus include the cryosupernatant, the cryoprecipitate (resuspended), the fractions I to V obtained by ethanol fractionation (according to the method of Cohn or of Kistler & Nitschmann), the supernatant and the precipitate obtained after precipitation with caprylic acid and/or caprylate, the eluates of chromatographies and the unadsorbed fractions of chromatography columns, and the filtrates.

Particularly advantageously, the starting plasma or plasma fraction subjected to the enrichment method of the invention comes from pooled blood plasmas from normal human subjects, without preliminary donor selection, in particular without donor selection based on their potential plasma anti-RSV immunoglobulin level. The starting plasma or plasma fraction subjected to the enrichment method of the invention thus advantageously comprises an anti-RSV immunoglobulin level similar or equal to the anti-RSV immunoglobulin level of a plasma derived from the pool of at least 1000 normal human donors selected randomly. The starting plasma or plasma fraction subjected to the enrichment method of the invention is advantageously not enriched with anti-RSV immunoglobulins in comparison with the anti-RSV immunoglobulin level of a plasma derived from the pool of at least 1000 donors selected randomly.

The starting plasma or plasma fraction subjected to the enrichment method of the invention contains human polyvalent immunoglobulins which can be immunoglobulin A (IgA), immunoglobulin E (IgE), immunoglobulin M (IgM) or immunoglobulin G (IgG). Advantageously, the starting plasma or plasma fraction contains essentially IgG regardless of their subclass (IgG1, IgG2, IgG3 and IgG4) and/or IgM.

Method for Purifying Immunoglobulins Hyper-Enriched with Anti-RSV Immunoglobulins:

The method of the invention preferentially uses as starting sample a plasma or a plasma fraction from pooled blood plasmas from normal human subjects, without preliminary donor selection, in particular without donor selection based on their potential plasma anti-RSV immunoglobulin level.

The method of the invention can typically be an independent method, dedicated only to the purification of anti-RSV immunoglobulins, and is thus advantageously carried out starting with plasma or a plasma fraction.

In this embodiment, the method according to the invention advantageously comprises the following steps:
providing a plasma or plasma fraction sample,
passing the plasma or plasma fraction sample by immunoaffinity chromatography through the matrix of the invention,
collecting the fraction adsorbed on the matrix of the invention corresponding to the immunoglobulin composition hyper-enriched with anti-RSV immunoglobulins.

Advantageously, the method according to the invention can further comprise, after the capture step using anti-RSV affinity chromatography, a dedicated step of depletion of certain immunoglobulins, in particular immunoglobulin E (IgE) and/or immunoglobulin (IgM), which may be involved in mechanisms of deleterious side effects in patients.

The method advantageously further comprises a subsequent step consisting in subjecting the immunoglobulin composition hyper-enriched with anti-RSV immunoglobulins to at least one virus inactivation and/or removal step.

In another embodiment of the invention, the method for obtaining anti-RSV immunoglobulins is carried out with an unused plasma fraction from an ancillary plasma protein purification process.

In this embodiment, the method according to the invention advantageously comprises the following steps:
providing a plasma or plasma fraction sample not used in a plasma protein purification process,
passing the plasma or plasma fraction sample by immunoaffinity chromatography through the matrix of the invention,
collecting the fraction adsorbed on the matrix of the invention corresponding to the immunoglobulin composition hyper-enriched with anti-RSV immunoglobulins.

Advantageously, the method according to the invention can further comprise, after the capture step using anti-RSV affinity chromatography, a dedicated step of depletion of certain immunoglobulins, in particular immunoglobulin E (IgE) and/or immunoglobulin (IgM), which may be involved in mechanisms of deleterious side effects in patients.

The method advantageously further comprises a subsequent step consisting in subjecting the immunoglobulin composition hyper-enriched with anti-RSV immunoglobulins to at least one virus inactivation and/or removal step.

In still another particular embodiment of the invention, the method for obtaining anti-RSV immunoglobulins is coupled with a polyvalent immunoglobulin G method in order to obtain, from the same starting plasma pool, both a concentrate of polyvalent immunoglobulin G and an immunoglobulin concentrate hyper-enriched with anti-RSV.

The method of the invention thus typically comprises a preliminary step of obtaining the plasma fraction, by ethanol fractionation and/or caprylic acid fractionation and/or chromatographic separation.

In this embodiment, the method according to the invention advantageously comprises the following steps:
providing a sample of a prepurified plasma fraction obtained by ethanol fractionation and/or caprylic acid fractionation and/or chromatographic separation,
passing the prepurified plasma fraction by immunoaffinity chromatography through the matrix of the invention,
collecting the fraction adsorbed on the matrix of the invention corresponding to the immunoglobulin composition hyper-enriched with anti-RSV immunoglobulins.

According to the invention, “chromatographic separation” refers to any chromatography step, whether ion-exchange (anion-exchange and/or cation-exchange) chromatography, mixed-mode chromatography, affinity chromatography (using ligands such as chemicals, antibodies, antibody fragments, aptamers).

According to the invention, chromatographic separation also refers to a single chromatography column, or to a set of chromatography columns, optionally utilizing the same type of support, in series (multiple-column mode, for example) or in parallel.

More precisely, the plasma fraction can be obtained by ethanol fractionation originally developed by Cohn et al. (Cohn et al., 1946. J. Am. Chem. Soc. 68, 459; Oncley et al., 1949, J. Am. Chem. Soc. 71, 541), or by chromatographic separation, as described for example in EP 0 703 922 and WO 99/64462, or by caprylic acid fractionation as described by Steinbuch et al. 1969, Arch Biochem Biophys. 134(2):279-84). Particularly preferred are the methods developed by the Applicant in patent applications WO 94/29334 and WO 02/092632, and most particularly that described in WO 02/092632. In this case, blood plasma, or an Ig-enriched blood plasma fraction, is subjected to caprylic acid fractionation (prepurification by precipitation of non-immunoglobulin contaminants), and a single chromatography on an anion-exchange resin support performed at alkaline pH. A virus inactivation treatment can be performed, preferably by solvent-detergent, as described by Horowitz in U.S. Pat. No. 4,764,369, optionally supplemented by a virus removal step by nanofiltration on 75N, 35N, 20N and/or 15N pore-size filters.

The Ig fraction thus collected is already concentrated, but can then undergo steps of further concentration by ultrafiltration, and of sterile filtration.

This concentrate is then subjected to an immunoaffinity chromatographic step through the matrix of the invention.

Advantageously, the method according to the invention can further comprise, preferably after the capture step using anti-RSV affinity chromatography, a dedicated step of depletion of certain immunoglobulins, in particular immunoglobulin A (IgA), and/or immunoglobulin E (IgE) and/or immunoglobulin M (IgM), which may be involved in mechanisms of deleterious side effects in patients. The immunoglobulins to be depleted are advantageously selected in particular according to the route of administration chosen for the immunoglobulin concentrate hyper-enriched with anti-RSV immunoglobulins and/or according to the contents of the starting plasma or plasma fraction sample.

In a particular embodiment, the method according to the invention comprises a step of anion-exchange chromatography, advantageously using TMAE-type chromatography, in order to deplete immunoglobulin M (IgM) from the immunoglobulin composition hyper-enriched with anti-RSV immunoglobulins.

The method advantageously further comprises a subsequent step consisting in subjecting the immunoglobulin composition hyper-enriched with anti-RSV immunoglobulins to at least one virus inactivation and/or removal step. The virus inactivation and/or removal step can consist of acidic pH treatment, solvent-detergent treatment, pasteurization, dry heat, nanofiltration and/or sterile filtration. Advantageously the method comprises a step of virus inactivation by solvent-detergent and a step of virus removal by nanofiltration. In a particular embodiment of the invention the method comprises a subsequent step of virus removal by nanofiltration.

The method can further comprise the subsequent steps consisting in adding one or more pharmaceutically acceptable stabilizers; and optionally freezing or lyophilizing the concentrate thus obtained.

Composition of Hyper-Enriched Immunoglobulins:

The hyper-enriched immunoglobulin composition obtained according to the enrichment method of the invention contains essentially human polyvalent immunoglobulins which can be immunoglobulin A (IgA), immunoglobulin E (IgE), immunoglobulin M (IgM) or immunoglobulin G (IgG). Advantageously, the immunoglobulin composition contains essentially IgG and/or IgM and/or IgA.

In a particular embodiment, the immunoglobulin composition according to the invention comprises essentially IgG, regardless of their subclass (IgG1, IgG2, IgG3 and IgG4).

The composition can be adapted by the person skilled in the art according to the route of administration selected and/or the mechanism of action sought. For administration via the intravenous route, for example, the composition preferentially contains essentially IgG and/or IgM, and is advantageously depleted of IgA. On the other hand, in other routes of administration such as the local route, the composition preferentially contains essentially IgG and/or IgA, the IgA allowing the recruitment of cells such as mucosal cells with alpha receptors.

The immunoglobulin composition obtained according to the enrichment method of the invention is an immunoglobulin composition hyper-enriched with anti-RSV immunoglobulins which has RSV-neutralizing activity and has therapeutic efficacy for the patient.

The expression “composition hyper-enriched with anti-RSV immunoglobulins” refers to an immunoglobulin composition comprising at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % RSV-neutralizing immunoglobulins.

In a particular embodiment of the invention, the composition hyper-enriched with anti-RSV immunoglobulins according to the invention advantageously comprises 10 to 100 wt % RSV-neutralizing immunoglobulins, more advantageously 30 to 100 wt % RSV-neutralizing immunoglobulins, preferentially 60 to 100 wt % RSV-neutralizing immunoglobulins.

In a particular embodiment of the invention, the anti-RSV immunoglobulin composition advantageously has an RSV-neutralizing activity superior to that of the blood plasma of normal human subjects, superior to that of the plasma of human subjects selected for their anti-RSV immunoglobulin titre, superior to that of RSV hyperimmune immunoglobulins (RSV-IVIG) such as Respigam® or RI-002 from Adma Biologics, and/or superior to that of anti-RSV monoclonal antibodies such as palivizumab (Synagis®).

In a particular embodiment of the invention, the anti-RSV immunoglobulin composition comprises at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % immunoglobulins specifically directed against at least one F protein epitope.

The composition hyper-enriched with anti-RSV immunoglobulins according to the invention advantageously comprises 10 to 100 wt % immunoglobulins specifically directed against at least one F protein epitope, even more advantageously 30 to 100 wt % immunoglobulins specifically directed against at least one F protein epitope, preferentially 60 to 100 wt % immunoglobulins specifically directed against at least one F protein epitope.

In another particular embodiment of the invention, the anti-RSV immunoglobulin composition comprises at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % immunoglobulins specifically directed against at least one F protein epitope in prefusion conformation.

The composition hyper-enriched with anti-RSV immunoglobulins according to the invention advantageously comprises 10 to 100 wt % immunoglobulins specifically directed against at least one F protein epitope in prefusion conformation, even more advantageously 30 to 100 wt % immunoglobulins specifically directed against at least one F protein epitope in prefusion conformation, preferentially 60 to 100 wt % immunoglobulins specifically directed against at least one F protein epitope in prefusion conformation.

In a particular embodiment of the invention, the anti-RSV immunoglobulin composition comprises at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % immunoglobulins specifically directed against at least one G protein epitope.

The composition hyper-enriched with anti-RSV immunoglobulins according to the invention advantageously comprises 10 to 100 wt % immunoglobulins specifically directed against at least one G protein epitope, even more advantageously 30 to 100 wt % immunoglobulins specifically directed against at least one G protein epitope, preferentially 60 to 100 wt % immunoglobulins specifically directed against at least one G protein epitope.

The composition hyper-enriched with anti-RSV immunoglobulins according to the invention advantageously comprises hyperneutralizing immunoglobulins directed against a plurality of RSV epitopes. Advantageously, the composition hyper-enriched with anti-RSV immunoglobulins according to the invention comprises anti-RSV immunoglobulins directed against at least 2, at least 3, at least 5, at least 10 different RSV epitopes.

In an advantageous embodiment of the invention, the composition hyper-enriched with anti-RSV immunoglobulins according to the invention comprises anti-RSV immunoglobulins directed against at least one F protein epitope and at least one G protein epitope.

In another particular embodiment of the invention, the composition enriched with anti-RSV immunoglobulins comprises a mixture of anti-RSV immunoglobulins and immunoglobulins directed against at least one other infectious agent. Advantageously the composition enriched with anti-RSV immunoglobulins thus comprises at least 30 wt % anti-RSV immunoglobulins, and at most 70 wt % immunoglobulins directed against at least one other infectious agent. The composition enriched with anti-RSV immunoglobulins can thus advantageously comprise at least 30 wt % anti-RSV immunoglobulins, and at most 70 wt % anti-tetanus and anti-hepatitis B immunoglobulins. In another advantageous embodiment, the composition enriched with anti-RSV immunoglobulins comprises at least 50 wt % anti-RSV immunoglobulins and at most 50 wt % immunoglobulins directed against at least one other infectious agent.

Advantageously, the composition enriched with anti-RSV immunoglobulins does not comprise more than 10, more than 7, more than 5, more than 3, more than 2 different infectious specificities.

Final Products:

In a particular embodiment of the invention, the immunoglobulin concentrate hyper-enriched with anti-RSV immunoglobulins for therapeutic use can be administered via any route of administration: ocular, nasal, intra-auricular, oral, sublingual, pulmonary, intraperitoneal, intravenous, percutaneous, subcutaneous, intramuscular, transmucosal, vaginal, rectal.

The Ig concentrates for therapeutic use are generally at concentrations ranging between 50 and 100 g/L. These concentrates are intended for clinical use and can in particular be injected via the intravenous route. In that respect, they must be secured and, if need be, contain excipients, such as stabilizers, compatible with this clinical use.

The Ig concentrates for therapeutic use can also be administered via the subcutaneous route. In this case, the concentration of the products is greater than or equal to 100 g/L, advantageously greater than or equal to 150 g/L. The Ig concentrates for therapeutic use can also be administered via the intramuscular route.

Advantageously, the immunoglobulin concentrate hyper-enriched with anti-RSV immunoglobulins comprises at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % polyvalent immunoglobulins capable of recognizing and/or of neutralizing RSV or one of its parts. In a particular embodiment of the invention, the immunoglobulin concentrate hyper-enriched with anti-RSV immunoglobulins comprises at least 30 wt % polyvalent immunoglobulins capable of recognizing and/or of neutralizing RSV or one of its parts. In another particular embodiment of the invention, the immunoglobulin concentrate hyper-enriched with anti-RSV immunoglobulins comprises at least 60 wt % polyvalent immunoglobulins capable of recognizing and/or of neutralizing RSV or one of its parts.

Advantageously, the immunoglobulin concentrate hyper-enriched with anti-RSV immunoglobulins advantageously comprises at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % RSV-neutralizing immunoglobulins. In a particular embodiment of the invention, the immunoglobulin concentrate hyper-enriched with anti-RSV immunoglobulins comprises at least 30 wt % RSV-neutralizing immunoglobulins. In a particular embodiment of the invention, the immunoglobulin concentrate hyper-enriched with anti-RSV immunoglobulins comprises at least 60 wt % RSV-neutralizing immunoglobulins.

In a particular embodiment of the invention, the anti-RSV immunoglobulin composition advantageously has an RSV-neutralizing activity superior to that of the blood plasma of normal human subjects, superior to that of the plasma of human subjects selected for their anti-RSV immunoglobulin titre, superior to that of RSV hyperimmune immunoglobulins (RSV-IVIG) such as Respigam® or RI-002 from Adma Biologics, and/or superior to that of anti-RSV monoclonal antibodies such as palivizumab (Synagis®).

Advantageously, the immunoglobulin concentrate hyper-enriched with anti-RSV immunoglobulins comprises at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % immunoglobulins specifically directed against at least one F protein epitope.

Particularly advantageously, the immunoglobulin concentrate hyper-enriched with anti-RSV immunoglobulins comprises at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % immunoglobulins specifically directed against at least one F protein epitope in prefusion conformation.

In a particular embodiment of the invention, the immunoglobulin concentrate hyper-enriched with anti-RSV immunoglobulins comprises at least 30 wt % immunoglobulins specifically directed against at least one F protein epitope in prefusion conformation. In a particular embodiment of the invention, the immunoglobulin concentrate hyper-enriched with anti-RSV immunoglobulins comprises at least 60 wt % immunoglobulins specifically directed against at least one F protein epitope in prefusion conformation.

Advantageously, the immunoglobulin concentrate hyper-enriched with anti-RSV immunoglobulins comprises at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % immunoglobulins specifically directed against at least one G protein epitope.

In a particular embodiment of the invention, the immunoglobulin concentrate hyper-enriched with anti-RSV immunoglobulins comprises at least 30 wt % immunoglobulins specifically directed against at least one G protein epitope. In a particular embodiment of the invention, the immunoglobulin concentrate hyper-enriched with anti-RSV immunoglobulins comprises at least 60 wt % immunoglobulins specifically directed against at least one G protein epitope.

The immunoglobulin concentrate hyper-enriched with anti-RSV immunoglobulins according to the invention advantageously comprises hyperneutralizing immunoglobulins directed against a plurality of RSV epitopes. Advantageously, the immunoglobulin concentrate hyper-enriched with anti-RSV immunoglobulins according to the invention comprises anti-RSV immunoglobulins directed against at least 2, at least 3, at least 5, at least 10 different RSV epitopes.

In a particular embodiment of the invention, the immunoglobulin concentrate hyper-enriched with anti-RSV immunoglobulins according to the invention comprises anti-RSV immunoglobulins directed against at least one F protein epitope and at least one G protein epitope.

In a particular embodiment of the invention, the immunoglobulin concentrate hyper-enriched with anti-RSV immunoglobulins comprises a mixture of anti-RSV immunoglobulins and immunoglobulins directed against at least one other infectious agent. Advantageously the immunoglobulin concentrate hyper-enriched with anti-RSV immunoglobulins thus comprises at least 30 wt % anti-RSV immunoglobulins, and at most 70 wt % immunoglobulins directed against at least one other infectious agent. The immunoglobulin concentrate hyper-enriched with anti-RSV immunoglobulins can thus advantageously comprise at least 30% anti-RSV immunoglobulins, and at most 70% anti-tetanus and anti-hepatitis B immunoglobulins.

In another advantageous embodiment, the immunoglobulin concentrate hyper-enriched with anti-RSV immunoglobulins comprises at least 50 wt % anti-RSV immunoglobulins and at most 50 wt % immunoglobulins directed against at least one other infectious agent.

Advantageously, the immunoglobulin concentrate hyper-enriched with anti-RSV immunoglobulins does not comprise more than 10, more than 7, more than 5, more than 3, more than 2 different infectious specificities.

The Ig concentrates enriched in anti-RSV Ig according to the invention are useful for treating RSV infection.

They can in particular be used for immunoprophylaxis in patients who are not infected but who are at risk: children, infants, hospitalized persons, immunocompromised persons, elderly persons.

They can also be used as a curative treatment by early administration. Advantageously, the immunoglobulin concentrate hyper-enriched with anti-RSV immunoglobulins according to the invention thus makes it possible to slow and/or to prevent the spread of the upper respiratory tract infection to the lower respiratory tract. In another advantageous embodiment, the Ig concentrate enriched with anti-RSV Ig according to the invention makes it possible to slow and/or to prevent the spread of RSV from cell to cell and/or to slow and/or to prevent the formation of syncytia.

Advantageously, the immunoglobulin concentrate hyper-enriched with anti-RSV immunoglobulins according to the invention enables the significant reduction of the viral load, normalized relative to a known reference, in the treated patient.

The immunoglobulin concentrate hyper-enriched with anti-RSV immunoglobulins according to the invention advantageously further enables the destruction of infected cells via the recruitment of effector cells by virtue of the ADCC functions of the immunoglobulins of the composition.

The treated patient is preferably a human being, regardless of age and sex. The therapeutic treatment of children and infants is particularly envisaged, as well as that of elderly, hospitalized or immunosuppressed persons.

### EXAMPLES

### Example 1: Affinity Chromatography Utilizing as Ligand hRSV-F Protein 11049-V08B

Preparation of the Anti-RSV Affinity Chromatography Matrix

The affinity ligand used is RSV-F protein 11049-V08B (Sino Biological Inc), a synthetic protein derived from an hRSV (RSS-2) DNA sequence comprising Met 1 to Thr 529, containing 518 amino acids after cleavage of the propeptide and having a predicted molecular mass of 58 kDa. In SDS-PAGE analysis and under reducing conditions, the apparent molecular masses are 45-55 kDa and 18 kDa. The RSV-F 11049-V08B ligand used for grafting is in postfusion conformation.

2 mL of NHS-Activated Sepharose™ 4 Fast Flow gel is loaded in a 1.1-cm diameter chromatography column and the buffers used are those recommended by the gel supplier. After washing (1 mM HCl at 4° C.) and equilibration with coupling buffer, 1.98 mg of F protein to be grafted is added to the gel and the whole is shaken. Blocking of the reactive groups on the gel is achieved by adding 6 mL of 0.5 M ethanolamine, 0.5 M NaCl, pH 8.3 buffer (6 mL), and the gel is shaken for 12 h. The coupled gel is washed by alternating 3 volumes of basic then acidic buffers: 0.1 M Tris-HCl pH 8.4 and 0.1 M acetate, 0.5 M NaCl pH 4, this cycle being repeated three times. The gel is then stored in 10 mM citrate, 0.5 M NaCl, 0.1 g/L sodium azide pH 6.6 buffer.

Coupling was evaluated at 98%, corresponding to a ligand density of 0.89 mg/mL of grafted gel.

TABLE 1
Coupling yield

| | Initial F | Desalted F protein | Coupling |
|---|---|---|---|
| | protein | for coupling | supernatant |
| Initial volume (mL) | 3 mL | 4.5 | 5.0 |
| Deposited sample | 0.66 | 0.44 | 0.039 |
| concentration (mg/mL) | | | |
| Amount deposited (mg) | 1.98 | 1.98 | 0.195 |
| Coupling yield | NA | NA | 98% |

In order to test its functionality, the gel undergoes a chromatography cycle with a polyvalent immunoglobulin sample (22.4 mL of sample ILP10, 98% IgG purity, concentration 9.45 g IgG/L, titred 0.0107 mg/mL anti-RSV). Contact time is 3.3 min. The following buffers are used:
fixing/washing: 10 mM citrate, pH 7, 0.5 M NaCl;
elution: 100 mM glycine-HCl, pH 2.57;

During the first run on this gel a well-defined elution peak is observed. The gel is thus functional and satisfactorily bound a portion of the immunoglobulins injected.

Execution of the Anti-RSV Affinity Chromatography

The sample used is a plasma fraction (ILP10) derived from the immunoglobulin purification process as described in the Applicant's patent application WO02092632. The purification process comprises the following steps:
Collecting a fraction I+II+III, obtained from ethanol-treated blood plasma
Caprylic acid precipitation
Solvent-detergent (Triton/TnBP) treatment
Anion-exchange chromatography (TMAE) at basic pH

The eluate of the anion-exchange chromatography at basic pH constitutes sample ILP10 no. 13606/10L02032 at 9.45 g/L IgG (99% IgG purity) and contains 0.11% (107 μg/mL) anti-RSV immunoglobulins.

The sample is equilibrated in the injection buffer by adding thereto a concentrated buffer solution of NaCl and trisodium citrate qs 0.05 M NaCl and 0.01 M trisodium citrate. The product is then passed through the column for 3.3 min (contact time). After washing, elution is carried out in 0.1 M glycine, 30% propylene glycol, pH 2.57 buffer. The eluate is immediately neutralized with 1 M Tris-HCl pH 9 buffer to increase the pH to about 6. The gel is then regenerated in order to detach the Ig not eluted with a 6 M guanidine pH 6 buffer. The collected fraction is also neutralized by adding 1 M Tris-HCl pH 9 to increase the pH to 6.

Results

Analytical assay of the fractions is carried out by the MSD (Meso Scale Discovery™) method.

The day before the assay, the plate is coated with F protein by adding 30 μL/well at 1.5 μg/mL F protein in PBS buffer, and the mixture is incubated overnight at 4° C. The plate is then washed three times with 200 μL of PBS, 0.1% Tween buffer. Saturation is then achieved by adding 150 μL per well of PBS, 3% BSA, and incubating 1 h at room temperature with shaking. The plate is washed again three times with 200 μL of PBS, 0.1% Tween buffer. The samples to be assayed are added in the amount of 25 μL per well, and incubated 2 h at room temperature with shaking. A new washing cycle identical to the preceding is carried out. The Sulfo-Tag anti-Human IgG conjugate is then added in the amount of 25 μL in each well, and incubated for 2 h at room temperature with shaking. A new washing cycle is carried out, then 150 μL per well of read buffer diluted by half in water is added. The plate is shaken moderately and read immediately afterwards.

The results obtained are presented in Table 2 below:

TABLE 2
Amount of anti-RSV Ig during the purification process

| | | | Ratio of | | | | |
|---|---|---|---|---|---|---|---|
| | | Amount | anti- | | | | |
| | Amount | of anti- | RSV | | | Eluted/ | Anti- |
| | of Total | RSV | IgG to | | Binding | bound | RSV Ig |
| | IgG | IgG | Total | Enrichment | yield | yield | results |
| | (mg) | (mg) | IgG (%) | factor | (%) | (%) | (%) |
| IgIV | 212 | 0.241 | 0.11% | 1.0 | | | 100% |
| starting | | | | | | | |
| material | | | | | | | |
| FNA | 177 | 0.055 | 0.03% | 0.3 | 69% | | 23% |
| | | | | | (166 μg) | | |
| Washing | 22 | 0.020 | 0.09% | 0.8 | | | 8% |
| Eluate | 0.268 | 0.092 | 34.1% | 301 | | 55% | 38% |
| Regeneration | 0.060 | 0.003 | 5.5% | 49 | | 1.8% | 1.2% |

A high proportion of anti-RSV immunoglobulins is bound to the chromatographic support by affinity to the hRSV-F protein (70%) which represents in this test 166 μg of anti-RSV immunoglobulins per mL of gel. The unadsorbed fraction and the wash fraction are depleted of anti-RSV immunoglobulins. The immunoglobulin fraction eluted from the support at acidic pH and in the presence of propylene glycol shows a 301-fold enrichment with anti-RSV immunoglobulins and the eluted proportion represents 55% of the proportion bound to the support.

It is noted that the regeneration fraction comprises a small amount of immunoglobulins but also comprises a high proportion of anti-RSV immunoglobulins which could be collected either by modifying the elution conditions of the chromatography (buffer composition, pH, etc.) or by passing the regeneration fraction through the column again in order to collect the anti-RSV immunoglobulins.

The test shows that affinity chromatography utilizing an F protein ligand in postfusion conformation grafted via NHS chemistry allows the purification of anti-RSV immunoglobulins with a 38% yield under the conditions tested (55% of the immunoglobulins bound to the support), corresponding to an enrichment factor of 301.

The composition obtained after affinity chromatography on F protein ligand in postfusion conformation thus comprises at least 30 wt % immunoglobulins specifically directed against at least one F protein epitope.

This composition is useful for preparing an immunoglobulin concentrate hyper-enriched with anti-RSV immunoglobulins comprising at least 30 wt % immunoglobulins specifically directed against at least one F protein epitope.

The fractions obtained were also analysed to determine the distribution of the various IgG subclasses.

The results are presented in Table 3 below:

TABLE 3
IgG subclass distribution

| | IgG1 | IgG2 | IgG3 | IgG4 |
|---|---|---|---|---|
| “Concentrated | 73% | 25% | 0.4% | 0.8% |
| eluate” fraction | | | | |
| “Concentrated | 75% | 25% | <limit of | <limit of |
| regeneration” | | | quantification | quantification |
| fraction | | | | |

### Example 2: Functional Test of the Anti-RSV Immunoglobulins Purified by Affinity Chromatography Utilizing as Ligand hRSV-F Protein 11049-V08B

Serum Neutralization Protocol

In order to measure the capacity of the fractions obtained to inhibit viral infection, a virus strain, hRSV(18)-cherry3, containing a reporter gene encoding a fluorescent protein is used on a Hep2 cell line. Fluorescence intensity is directly representative of viral replication. The day before the test, the cells are subcultured in a 96-well plate at 0.5×10⁶ cells/mL and incubated at 37° C. in 7% CO₂. The day of the test, the positive control monoclonal antibodies (Synagis™, solution of monoclonal antibodies directed against RSV) and the negative control monoclonal antibodies (Humira™, solution of anti-TNF monoclonal antibodies) as well as the fractions to be tested are diluted in MEM and 70 μL is deposited in the wells of a 96-well plate. The virus is also diluted 1:25 in MEM and 70 μL is deposited in the wells containing the antibody solutions. Homogenization is achieved by aspiration/ejection. The mixture is then incubated for 1 h at 37° C. in 7% CO₂. For infection of the cells, the cell supernatant is aspirated, 100 μL of the Ig/virus mixture is quickly deposited and incubation is carried out at 37° C. in 7% CO₂ for 36 to 48 hours. Development was carried out by measurement of fluorescence by exciting at 580 nm and by reading at 620 nm. The values are normalized to the “100% infection” control where the virus alone is contacted with the cells, and the “0% infection” control corresponds to the cells contacted with MEM alone. An IC50 was determined using the curves of dose-response as a function of antibody concentration.

Results

The fractions enriched with anti-RSV immunoglobulins obtained in Example 1 (eluate and regeneration) are concentrated, formulated in buffer (mannitol, glycine, Tween 80) and subjected to a serum neutralization test.

TABLE 4
Serum neutralization test

| | | | | Concentrated | | Concentrated |
|---|---|---|---|---|---|---|
| | SYNAGIS* | IVIG | Eluate | eluate | Regeneration | regeneration |
| LogIC50 | 2.16 | 4.35 | 1.51 | 1.45 | ~2.407 | 1.25 |
| ng/mL | | | | | | |
| IC50 | 145.3 | 22338 | 32.63 | 28.32 | ~255.5 | 17.82 |
| ng/mL | | | | | | |
| Relative | 0.65 | 100 | 0.15 | 0.13 | NA | 0.08 |
| IC50 | | | | | | |

*The Synagis IC50 value was averaged from 3 tests.

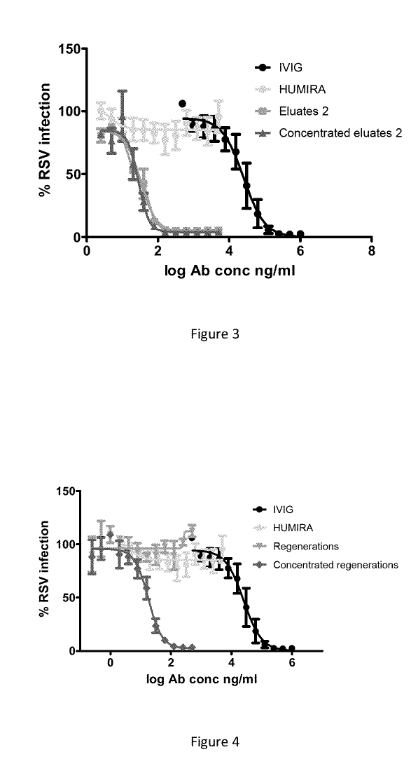

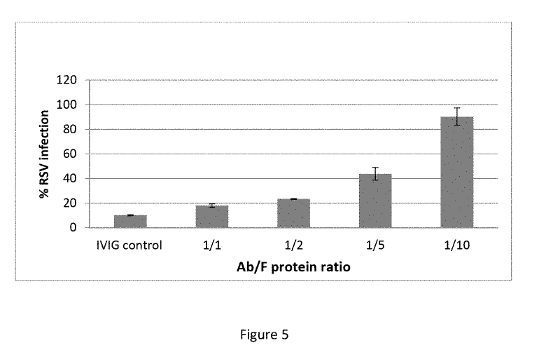

As summarized in FIGS. 3, 4 and 5, the serum neutralization activity observed in the purified fractions (IC50 at 28 ng/mL) is quite superior to that of a commercial polyvalent immunoglobulin solution (IVIG TEG 22×10³ ng/mL) and superior to that of SYNAGIS (145 ng/mL). The chromatography method used thus makes it possible to obtain anti-RSV immunoglobulins having an enhanced in vitro functional activity. This serum neutralizing activity is also detectable in the regeneration fractions with a lower IC50.

The immunoglobulin concentrate hyper-enriched with anti-RSV immunoglobulins obtained by affinity chromatography thus has a neutralizing activity allowing its use in the treatment of RSV infection.

### Example 3: Affinity Chromatography Utilizing as Ligand RSV-G Protein, Strains A (11070-V08H2, SB) or B (13029-V08H, SB)

Preparation of the Anti-RSV Affinity Chromatography Matrix

The affinity ligand used is the RSV-G protein
strain A (ref. 11070-V08H2, SB), a synthetic protein derived from an hRSV (strain rsb1734) DNA sequence comprising Asn 66 to Arg 297, containing 242 amino acids after cleavage of the propeptide and having a predicted molecular mass of 26.3 kDa. In SDS-PAGE analysis and under reducing conditions, the apparent molecular mass is, because of high glycosylation, between 60 kDa and 90 kDa.
strain B (ref. 13029-V08H, SB), a synthetic protein derived from an hRSV (strain 036633-1) DNA sequence comprising His 67 to Ala 299, containing 244 amino acids after cleavage of the propeptide and having a predicted molecular mass of 27 kDa. In SDS-PAGE analysis and under reducing conditions, the apparent molecular mass is, because of high glycosylation, between 80 kDa and 90 kDa.

Coupling is carried out according to the same protocol as that used in Example 1. Three columns are prepared:
gel G-A, corresponding to a strain-A G-protein density of 1 mg/mL of grafted gel,
gel G-B, corresponding to a strain-B G-protein density of 1 mg/mL of grafted gel,
gel G-AB, corresponding to a 50% gel G-A and 50% gel G-B mixture.

Execution of the Anti-RSV Affinity Chromatography

The sample used is a plasma fraction derived from the immunoglobulin purification process as described in the Applicant's patent application WO02092632. The purification process comprises the following steps:
Collecting a fraction I+II+III, obtained from ethanol-treated blood plasma
Caprylic acid precipitation
Solvent-detergent (Triton/TnBP) treatment
Anion-exchange chromatography

The anion-exchange chromatography eluate constitutes the IgG sample (≥98% IgG purity) and contains 0.04% anti-RSV immunoglobulins. Contact time is 3 min. The following buffers are used:
fixing/washing: 20 mM phosphate, pH 7;
elution: 100 mM glycine-HCl, pH 2.5.

Results

Analytical assay of the fractions is carried out by the nephelometric method (total IgG) and by CFCA assay using Biacore (anti-RSV Ig) according to the following protocol:
Immobilization of G protein variants A and B on a CM5 sensor chip
Injection of the fractions derived from the chromatography steps
Measurement of the rate of diffusion of the anti-RSV IgG from the running buffer to the interaction surface
Calculation of the absolute concentration of anti-RSV IgG in the various fractions based on the measured rate of diffusion and the diffusion properties of the IgG.

TABLE 5
Amount of anti-RSV Ig during the purification process on gel G-A

| | | | Ratio of | | |
|---|---|---|---|---|---|
| | | | anti-RSV | | |
| | Amount | Amount of | IgG to | | Anti-RSV |
| | of Total | anti-RSV | Total | Enrichment | Ig results |
| | IgG (μg) | IgG (μg) | IgG (%) | factor | (%) |
| IgIV | 133770 | 31 | 0.023% | — | 100% |
| starting | | | | | |
| material | | | | | |
| FNA | 12810 | ND | — | — | — |
| Eluate | 337 | 8 | 2.5% | 109 | 26.8% |

TABLE 6
Amount of anti-RSV Ig during the purification process on gel G-B

| | | | Ratio of | | |
|---|---|---|---|---|---|
| | | | anti-RSV | | |
| | Amount | Amount of | IgG to | | Anti-RSV |
| | of Total | anti-RSV | Total | Enrichment | Ig results |
| | IgG (μg) | IgG (μg) | IgG (%) | factor | (%) |
| IgIV | 133770 | 21 | 0.015% | — | 100% |
| starting | | | | | |
| material | | | | | |
| FNA | 144900 | ND | — | — | — |
| Eluate | 349 | 8 | 2.4% | 153 | 26.2% |

TABLE 7
Amount of anti-RSV Ig during the
purification process on gel G-AB

| | | | Ratio of | | |
|---|---|---|---|---|---|
| | | | anti-RSV | | |
| | Amount | Amount of | IgG to | | Anti-RSV |
| | of Total | anti-RSV | Total | Enrichment | Ig results |
| | IgG (μg) | IgG (μg) | IgG (%) | factor | (%) |
| IgIV | 133770 | 52 | 0.04% | — | 100% |
| starting | | | | | |
| material | | | | | |
| FNA | 124000 | ND | — | — | — |
| Eluate | 288 | 14 | 4.8% | 124 | 26.7% |

NB: the experimental design enabled us to note that the anti-RSV IgG purified on G protein variant A also interacted with G protein variant B and vice versa (anti-varB on variant A) suggesting that a cross-reactivity exists between the anti-RSV polyclonal antibodies for the two G protein variants.

A high proportion of anti-RSV immunoglobulins is bound to the chromatographic support by affinity to the hRSV-G protein, regardless of the strain, which represents in this test at least 6.7 μg of anti-RSV immunoglobulins per mL of gel. The unadsorbed fraction and the wash fraction are depleted of anti-RSV immunoglobulins. The immunoglobulin fraction eluted from the support at acidic pH and in the presence of propylene glycol shows a minimum 109-fold enrichment with anti-RSV immunoglobulins.

The test shows that affinity chromatography utilizing as ligand the G protein of strain A or B grafted via NHS chemistry allows the purification of anti-RSV immunoglobulins with a yield of at least 26.2% under the conditions tested, corresponding to a minimum 109-fold enrichment factor.

The composition obtained after affinity chromatography on G protein ligand thus comprises at least 30 wt % immunoglobulins specifically directed against at least one G protein epitope.

This composition is useful for preparing an immunoglobulin concentrate hyper-enriched with anti-RSV immunoglobulins comprising at least 30 wt % immunoglobulins specifically directed against at least one G protein epitope.

1-18. (canceled)

19. A method for preparing an immunoglobulin (Ig) concentrate useful for treating a respiratory syncytial virus (RSV) infection comprising a step consisting in subjecting an Ig composition derived from blood plasma to affinity chromatography utilizing an RSV-specific ligand, wherein the RSV-specific ligand is an RSV protein, or a variant thereof or an antigenic fragment thereof, and comprising the subsequent step consisting in collecting the fraction adsorbed on the affinity matrix and subjecting it to at least one virus inactivation and/or removal step.

20. The method for preparing a syncytial immunoglobulin (Ig) concentrate according to claim 19, wherein the RSV protein is an RSV surface protein, or a variant thereof or an antigenic fragment thereof.

21. The method according to claim 19, wherein the RSV-specific ligand is an RSV SH protein and/or G protein and/or F protein, or a variant thereof or an antigenic fragment thereof.

22. The method according to claim 19, wherein the RSV-specific ligand is an RSV F protein, in prefusion conformation, or a variant thereof or an antigenic fragment thereof.

23. The method according to claim 21, wherein the RSV F protein contains the mutations S155C, S290C, S190F and V207L.

24. The method according to claim 21, wherein the RSV F protein comprises the fragments F24-136, F164-315, F283-402, F403-524, F167-201, F235-275, and/or F478-512.

25. The method according to claim 19, wherein the RSV-specific ligand is an RSV G protein or a variant thereof or an antigenic fragment thereof.

26. The method according to claim 19, wherein the blood plasma utilized consists of pooled blood plasmas from normal human subjects, without preliminary donor selection.

27. The method according to claim 19, wherein the affinity matrix is made up of a polymer gel.

28. The method according to claim 19, comprising a preliminary step of obtaining the Ig composition from blood plasma, by ethanol fractionation and/or caprylic acid fractionation and/or chromatographic separation.

29. The method according to claim 19, further comprising a step of adding one or more pharmaceutically acceptable stabilizers.

30. The method according to claim 19, wherein the immunoglobulin (Ig) concentrate consists of a polyvalent immunoglobulin concentrate capable of neutralizing RSV or one of its parts.

31. The immunoglobulin (Ig) concentrate hyper-enriched with anti-RSV immunoglobulins obtainable by the method according to claim 19.

32. An immunoglobulin (Ig) concentrate hyper-enriched with anti-RSV immunoglobulins, comprising at least 10%, at least 20%, at least 30%, at least 40%, at least 50%, at least 60%, at least 65%, at least 70%, at least 75%, at least 80%, at least 85%, at least 90%, at least 95%, at least 96%, at least 97%, at least 98%, at least 99% polyvalent immunoglobulins capable of recognizing and/or of neutralizing RSV or one of its parts.

33. An immunoglobulin (Ig) concentrate hyper-enriched with anti-RSV immunoglobulins comprising at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % RSV-neutralizing immunoglobulins.

34. An immunoglobulin (Ig) concentrate hyper-enriched with anti-RSV immunoglobulins comprising at least 10 wt %, at least 20 wt %, at least 30 wt %, at least 40 wt %, at least 50 wt %, at least 60 wt %, at least 65 wt %, at least 70 wt %, at least 75 wt %, at least 80 wt %, at least 85 wt %, at least 90 wt %, at least 95 wt %, at least 96 wt %, at least 97 wt %, at least 98 wt %, at least 99 wt % immunoglobulins specifically directed against at least one F protein epitope in prefusion conformation.

35. The immunoglobulin (Ig) concentrate hyper-enriched with anti-RSV immunoglobulins according to claim 31, for use in the treatment of RSV infection.

36. The method of claim 27, wherein the polymer gel is an organic polymer.

37. The method according to claim 20, wherein the RSV-specific ligand is an RSV SH protein and/or G protein and/or F protein, or a variant thereof or an antigenic fragment thereof.

38. The method according to claim 29, further comprising a step of freezing or lyophilizing the concentrate thus obtained.


SEQUENCE LISTING (5 sequences)

SEQ ID NO: 1 - protein, 574 aa, respiratory syncytial virus (RSV)
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKTTPVTLSKDQLSGINNIAFSK

SEQ ID NO: 2 - protein, 574 aa, respiratory syncytial virus (RSV)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 3 - protein, 574 aa, respiratory syncytial virus (RSV)
MELPILKTNAITAILAAVTLCFASSQNITEEFYQSTCSAVSKGYLSALRTGWYTSVITIELSNIKENKCNGTDAKVKLIKQELDKYKSAVTELQLLMQSTPATNNRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKDRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 4 - protein, 297 aa, respiratory syncytial virus (RSV)
MSKNKDQRTAKTLERTWDTLNHLLFISSCLYKLNLKSVAQITLSILAMIISTSLIIVAIIFIASANHKITSTTTIIQDATNQIKNTTPTYLTQNPQLGISPSNPSDITSLITTILDSTTPGVKSTLQSTTVGTKNTTTTQAQPNKPTTKQRQNKPPSKPNNDFHFEVFNFVPCSICSNNPTCWAICKRIPNKKPGKRTTTKPTKKPTPKTTKKGPKPQTTKSKEAPTTKPTEEPTINTTKTNIITTLLTSNTTRNPELTSQMETFHSTSSEGNPSPSQVSITSEYPSQPSSPPNTPR

SEQ ID NO: 5 - protein, 299 aa, respiratory syncytial virus (RSV)
MSKHKNQRTARTLEKTWDTLNHLIVISSCLYRLNLKSIAQIALSVLAMIISTSLIIAAIIFIISANHKVTLTTVTVQTIKNHTEKNITTYLTQVPPERVSSSKQPTTTSPIHTNSATTSPNTKSETHHTTAQTKGRTTTSTQTNKPSTKPRLKNPPKKPKDDYHFEVFNFVPCSICGNNQLCKSICKTIPSNKPKKKPTIKPTNKPTTKTTNKRDPKTPAKTTKKETTTNPTKKPTLTTTERDTSTSQSTVLDTTTLEHTIQQQSLHSTTPENTPNSTQTPTASEPSTSNSTQNTQSHA